(19) United States
(12) Patent Application Publication
(10) Pub. No.: US 2005/0149055 A1
(43) Pub. Date: Jul. 7, 2005

(54) Device that facilitates the handling of an animal cornea and, in particular, a human cornea

(76) Inventors: Daniel Briday, Dommartin (FR); Guy Boucher, Sassenage (FR)

Correspondence Address: SUGHRUE MION, PLLC, 2100 PENNSYLVANIA AVENUE, N.W., SUITE 800, WASHINGTON, DC, 20037, US

(21) Appl. No.: 10/503,474

(22) PCT Filed: Feb. 4, 2003
(86) PCT No.: PCT/FR03/00339

(30) Foreign Application Priority Data
Feb. 4, 2002 (FR) 02/01260

(51) Int. Cl. A61F 9/00

(52) U.S. Cl. 606/107

(57) ABSTRACT

A device for facilitating the handling of an animal cornea, in particular a human cornea, seeking to optimize the preservation of corneal cells. The device is inserted into various flasks and media used before performing a corneal graft. The invention relates to a device which minimizes the contact areas during preservation of the cornea, and prevents it from being handled directly so as to preserve an optimum cell quality before performing the graft. The device is constituted by a support (1) on which the cornea (11) is disposed, said support is fixed to a handling element (9) by clips (4).

The present invention relates to a device for facilitating the handling of an animal cornea, in particular a human cornea, and seeks to optimize the preservation of corneal cells until grafting is performed. The device of the invention is inserted into various flasks containing the liquid media used before performing a corneal graft.

In the case of humans, grafting a cornea onto a patient firstly requires the cornea to be taken from a dead person. The removed cornea is then inserted by the surgeon into a first flask containing a “sample-taking” medium. It is then inspected under a microscope, and then inserted into a second flask containing a preservative liquid. Finally, a few hours before performing the graft, a third flask is used containing a deturgescent medium, i.e. a medium for reducing the swelling of the cornea.

Currently, in the sample-taking flask, the cornea is disposed freely in the solution. In the preservation and deturgescent flasks the technique is to hold the cornea in suspension at the end of a thread that is fixed on the periphery of the cornea by piercing it with a needle. This current technique presents numerous drawbacks:
handling is awkward;
the cornea must be handled with sterilized or disposable instruments;
the risk of contamination and/or of infection is high;
multiple contact against the walls of the flasks can spoil the corneal cells;
stagnation of the cornea at the bottom of the sample-taking flask presents the risk of the cornea adhering to the walls of the flask, and implies contact with the degenerate cells stagnating at the bottom of a flask.

Document EP-0 262 766 discloses a cornea support device formed on the stopper of a flask. That device requires special modified flasks to be used, with standard flasks not being adaptable to this system.

An object of the present invention is to provide a device which does not reproduce the drawbacks of the prior art.

A particular object of the present invention is to provide such a device which is simple and inexpensive to manufacture, to assemble, and to use, while making it possible to use standard non-modified flasks.

The present invention therefore provides a device for facilitating the handling of a human cornea, seeking to optimize the preservation of corneal cells in various flasks and media used before performing the corneal graft, the device being characterized in that it includes a support on which the cornea is disposed, and a handling element for holding said support and for extracting it from the various flasks and media.

Advantageously, said device is entirely independent from the flasks and/or from their stoppers.

Without being compressed, the cornea advantageously remains contained in a space reduced to a minimum.

The surfaces in contact with the cornea are advantageously minimized.

The support is advantageously held by the handling element in such a manner that the cornea is positioned above the bottom of the flask, where degenerate cells stagnate, by means of support elements such as prongs.

The support advantageously presents at least one orifice which allows light to pass directly onto the cornea during analysis under a microscope.

Other characteristics and advantages of the present invention appear more clearly from the following detailed description of a particular embodiment of the invention, given by way of non-limiting example, and made with reference to the accompanying drawing, in which:

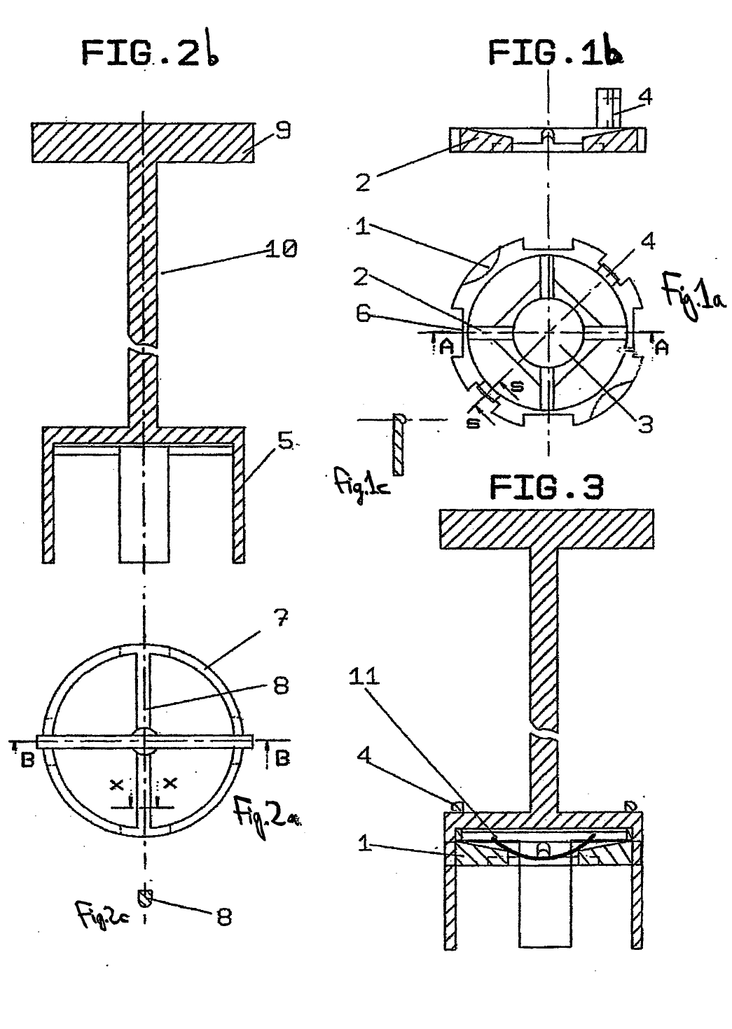

FIG. 1a is a plan view of the cornea support constituting an embodiment of the present invention;

FIG. 1b is a section view on line A-A of FIG. 1a;

FIG. 1c is a section view on line S-S of FIG. 1a;

FIG. 1a is a plan view of the corresponding handling element;

FIG. 1b is a section view on line B-B of FIG. 2a;

FIG. 2c is a section view on line X-X of FIG. 2a; and

FIG. 3 is a section view of the assembled device.

With reference to the drawings, and in an advantageous embodiment of the present invention, the device includes a support 1, having approximately the shape of a wheel with spokes 2, and pierced at its center.

The spokes 2 slope downwards (in the position shown in FIGS. 1b and 3) so as to center the cornea 11, and they preferably present a rounded profile so as to minimize contact area.

The height of the support 1 is preferably limited by the height of the transparent sterile boxes into which it is inserted, together with the cornea, during analysis under a microscope, in order to avoid touching the cornea. The central hole 3 of the support 1 allows light from the microscope to pass, and allows the cornea to be viewed directly.

A handling element is provided to handle the support 1. The handling element 9 can be fixed in removable manner to said support 1. For example, two fixing clips 4 can be used to hold the support 1 on the handling element 9. Other fixing means can also be envisaged. The handling element advantageously includes prongs 5 which come to slide into notches 6 of the support 1, so as not to hinder the handling of the support. Advantageously, the prongs are long enough to hold the support 1 above the bottom of the flask where degenerate cells stagnate.

The handling element 9 of the device of the invention can also present a wheel 7 with spokes 8 having a rounded profile so as to minimize contact area. The handling element can include a T-shaped rod 10 of length and width that are adapted to standard flasks so as to enable the device to be centered at the neck of said flask.

Advantageously, the device of the invention is therefore completely independent from flasks and/or from their stoppers, thereby making it possible to use existing standard flasks that are normally used without the device of the invention. This is very profitable since the use of special modified flasks is avoided.

The device is preferably made of plastics material and can be manufactured in a single molding operation by injection on a press. A metal device, or a device made of other materials, such as ceramics, can also be envisaged.

The device is preferably sterilized and disposable.

The device of the invention is particularly adapted to handling a human cornea before performing a graft.

Thus, the device of the invention facilitates handling, reduces the contact areas with the cornea, and eliminates finger contact. In particular, it makes it possible:
to hold the support at a determined height;
to center the device in the flask; and
to insert and extract the support cleanly from the flask, without any risk of damaging the cornea.

Although the present invention is described above with reference to a particular embodiment thereof, naturally it is not limited to that embodiment. Thus, the spoked-wheel shape could be modified. Other modifications could also be envisaged by the person skilled in the art without going beyond the ambit of the present invention defined by the accompanying claims.

1. A device for facilitating the handling of a human cornea, seeking to optimize the preservation of corneal cells in various flasks and media used before performing the corneal graft, the device being characterized in that it includes a support (1) on which the cornea (11) is disposed, and a handling element (9) for holding said support and for extracting it from the various flasks and media.

2. A device according to claim 1, in which said device is entirely independent from the flasks and/or from their stoppers.

3. A device according to claim 1, characterized in that, without being compressed, the cornea (11) remains contained in a space reduced to a minimum.

4. A device according to claim 1, characterized in that the surfaces in contact with the cornea (11) are minimized.

5. A device according to claim 1, characterized in that the support (1) is held by the handling element (9) in such a manner that the cornea (11) is positioned above the bottom of the flask, where degenerate cells stagnate, by means of support elements such as prongs (5).

6. A device according to claim 1, characterized in that the support (1) presents at least one orifice (3) which allows light to pass directly onto the cornea (11) during analysis under a microscope.
